(19) United States
(12) Patent Application Publication
(10) Pub. No.: US 2004/0054250 A1
(43) Pub. Date: Mar. 18, 2004

(54) Constructive arrangement interoduced in a therapeutical device with electromagnetical features

(76) Inventors: Persio Campos Benincasa, Campinas (BR); Ignacio Gorre, Campinas (BR)

Correspondence Address: SHAPIRO COHEN, P.O. BOX 3440, STATION D, OTTAWA, ON, K1P6P1, CA

(21) Appl. No.: 10/457,477

(22) Filed: Jun. 10, 2003

(30) Foreign Application Priority Data
Sep. 17, 2002 (BR) BR-MU8202176-7

(51) Int. Cl. A61N 2/00

(52) U.S. Cl. 600/9

(57) ABSTRACT

A therapeutically device with electromagnetic features constituted of a set comprising an anatomic and orthopedic mattress sufficiently stable for the backbone to be kept in an orthopedically acceptable position, provided with pillow top, which is an extra layer attached to the higher and/or lower part of the mattress, so to assure better comfort and sweetness, both covered by personalized Jacquard cloth with antiallergenic, antimite and antimould treatment. The mattress comprises various layers, i.e.: medium density polyurethane foams, agglomerated foam, high density polyurethane foam, light and undeformable expanded polypropylene or expanded polyethylene plate and special foam, including natural latex, blue dye, hydrated alumina, calcite, ricinoleate and an acrylic polymer, smooth and resistant covering, composing in this fashion a lighter structure with a lower quantity of layers and consequently less assembling stages in the production line. Pillow top to be used on the higher and/or lower part of the mattress comprises an extra foam layer overlapped by a permeable therapeutical profiled material provided with protuberances providing a smooth touch, causing body to relax, being said protuberances interspersed with magnet tablets which, together with the acrylic or polyester mantle layer overlaying it and which is treated and impregnated with powdered tourmaline, generate an electromagnetic force field with the capacity to naturally promote the perfect balance of body and mind health, by acting directly on blood circulation, eliminating toxins by breaking the water molecule group, causing body liquids to become weakly alkaline, sending oxygen to cells, speeding up the cell renewal process and helping to balance organ operations, combating physical fatigue, muscle pains and stress, providing deeper sleep, eliminating the daily fatigue and renewing energies. Said impregnation of powdered tourmaline on the acrylic, polyester or general cloth mantle is done by mixing it to the butadiene-styrene copolymer latex added to a ricinoleate solution (Sulfopon®), being said mixing sprayed so to be homogeneously spread over its surface and, after drying, said mixing is impregnated, thus becoming a part of the mantle or cloth, which now has electromagnetic features by means of a simple process with low cost for industrial feasibility.

### BACKGROUND OF THE INVENTION

1. Field of the Invention

The present invention deals with a therapeutical device with electromagnetic features.

The present invention relates to a therapeutical device with electromagnetic features having as its object the presentation of new, exclusive and functional features to be used in the therapeutical area to rest the body while sleeping, protecting the backbone and providing full relax, characterized by being a set composed by a sufficiently stable mattress for the backbone to be kept in an orthopedically acceptable position, provided with a pillow top, which is an extra layer attached to the higher and/or lower part of the mattress, so to assure better comfort and sweetness, which is composed by an extra foam layer overlapped by a permeable therapeutical profiled material provided with protuberances interspersed with magnet tablets, being overlaid by an acrylic or polyester mantle, treated and impregnated with powdered tourmaline, thus generating an electromagnetic field which is able to naturally promote the perfect balance of body and mind health, besides attempting to obtain a light set to facilitate sheet exchange not overloading the backbone, meeting the requirements of utility and satisfying technical conditions regarding strength, safety, practicability, quality and performance, so to characterize it as a low cost model for industrial feasibility, able to meet the actual requirements and needs of the consumer market

2. Description of the Prior Art

A few kinds of orthopedic and anatomic mattresses are currently known and conventionally used, as well as magnetic mattresses, which only contain small magnets spread over it so to awake the therapeutic capacity originally hidden in men, by means of an electromagnetic induction process.

Scientific assays show that human health is conditioned to magnetic power, inhibiting atrophies of the self-defensive system of the organism, by activating forces stimulating and encouraging the human body to improve more and more its self-defense capacity against diseases.

Researchers admit the existence of a magnetic field on the Earth's surface, as well as the existence of a modem syndrome of magnetic scarceness, which data point out that there is a trend to the gradual and stepwise reduction of the Earth magnetism, until arriving at a defective stage, being said scarceness increased by the high number of iron and cement buildings currently existing, as well as the use of electric and electronic devices and vehicles isolating people from more direct contact with magnetic forces, thus exerting deep influence over the health and state of mind of human beings.

Therefore, when we sleep on a magnetic therapeutic mattress, it acts as if we were resting over a magnetic field, causing the restitution of the organism's magnetism and thus the electrolytic dissolution process, generating electrical current, thus increasing blood ionization and improving blood circulation, so to reanimate the organism and its self-defense.

As known, magnetic therapeutical mattresses usually comprise a foam on which small spread over magnets are glued, with the purpose to form a uniform magnetic field causing electromagnetic induction.

However, said tablet-shaped magnets become cumbersome or unviable, and their application need to be duly studied so not to cause discomfort to the user, since they are rigidly defined and therefore need to be safely fitted and/or glued, thus avoiding its detachment, and homogeneously distributed, so to obtain a regular electromagnetic wave field.

There are various materials presenting electromagnetic features, such as granite torches and a few ceramic oxides, among others, but all of them with less significant potential in comparison to tourmaline, which is considered the strongest source of high energy radiation for its scientifically proven and recognized electromagnetic properties, such as: piezoelectricity, pyroelectricity, infrared radiation emission and others.

Tourmaline is a silicate mineral, a boron and aluminum complex with magnesium, iron and other alkaline metals, and crystal structure with multiple stages, being named electric stone for having permanent electrical poles which are maintained even when transformed into very small crystals, disappearing only after submitting them to a temperature over 1000° C.

Tourmaline crystals may therefore be molded, so to take the form of tablets, or incorporated to plastic material or to cloth or mantle wires in general for application in various forms to provide their use near the body, sending in the electromagnetic waves of tourmaline, activating organism cells, improving blood circulation, eliminating toxins by breaking the water molecule group and causing body liquids to become weakly alkaline, by helping to balance the operation of organs.

Bearing in mind the results of numerous researches, this invention seeks to provide a therapeutical device with electromagnetic features attempting to provide betterments with a product with high performance and trustfulness, promoting and distinguishing it among their similars, for being a set comprising an anatomic and orthopedic mattress with five layers, which is sufficiently stable for the backbone to be kept in the right position, provided with pillow top, which is an extra layer attached to the higher and/or lower part of the mattress, so to assure better comfort and sweetness.

### SUMMARY OF THE INVENTION

The present invention provides a pillow top, which comprises an extra foam layer overlapped by a permeable therapeutical profiled material provided with protuberances interspersed with magnet tablets, being then overlaid by a polyester or acrylic mantle which is treated and impregnated with powdered tourmaline, thus generating an electromagnetic force field, which is able to naturally promote the perfect balance of health of body and mind, besides attempting to obtain a light set to facilitate sheet change not overloading the backbone.

Said impregnation of the acrylic, polyester or general cloth mantle as used on the higher part of the pillow top with the powdered tourmaline happens by mixing it to the butadiene-styrene copolymer latex added to a ricinoleate solution containing 39-45% of soap (Sulfopon®), being said mixing sprayed so to homogeneously spread it over its surface and, after drying, said mixing is impregnated, thus becoming a part of the mantle or cloth, which now has electromagnetic features by means of a simple process with low cost for industrial feasibility.

Another important feature is that the mattress itself comprises various layers of foam and agglomerated foam with various densities, with an EPP plate in its intermediate region, i.e. a expanded polypropylene or expanded polyethylene plate which main advantage of use of said material is the fact of being a light and very durable material, since it is provided with memory, so to avoid deformation and wear usually verified with other mattresses making use of conventional foams, besides reducing the quantity of layers and consequently the assembly stages in the production line.

Therefore we have a highly efficient model, which is notable for its structural and functional simplicity, thus offering to the user an additional efficient option in the market of similars.

### BRIEF DESCRIPTION OF THE DRAWING

FIG. 1 is a perspective view of the pillow top set and mattress with a partial cut showing internal layers composing each part

### DETAILED DESCRIPTION OF THE INVENTION

The invention will be described for the purposes of illustration only in connection with certain embodiments; however, it is to be understood that other objects and advantages of the present invention will be made apparent by the following description of the drawings according to the present invention. While a preferred embodiment is disclosed, this is not intended to be limiting. Rather, the general principles set forth herein are considered to be merely illustrative of the scope of the present invention and it is to be further understood that numerous changes may be made without straying from the scope of the present invention.

A therapeutical device with electromagnetic features comprising in combination:

an anatomic, orthopedic mattress that is sufficiently stable for the backbone to be kept in an orthopedically acceptable position;

at least one electromagnetic pillow top attached to one side of the mattress, the mattress and the pillow top both being covered by a cloth provided with antiallergenic, antimite and antimould properties;

the mattress comprising a medium density polyurethane foam layer, an agglomerated foam layer, a high density polyurethane foam layer, a light and undeformable plate, a natural latex foam layer, and a smooth and resistant covering; and

the pillow top comprising an extra foam layer overlapped by a permeable therapeutical profiled material with protuberances being interspersed with magnet tablets, the extra foam layer being overlapped by a mantle layer impregnated with powdered tourmaline.

Numerous advantages and features of the invention have been explained in the above description, together with details of the structure and function of the invention, elements of novelty are pointed out by claims making part of the privilege application, but it is only illustrative and changes can be made on details and arrangements of parts, always within the principle of the arrangement introduced by the invention up to the full extension as indicated by the knowledge of the claims.

As described and illustrated, it is a new concept of therapeutically device with electromagnetic features, which technical, constructive and functional features are fully different from those pertaining to the state of the art.

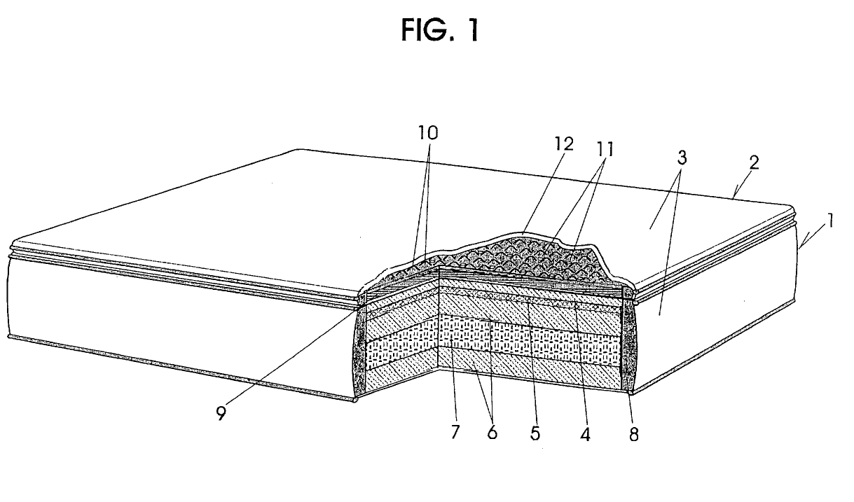

FIG. 1 shows a therapeutically device with electromagnetic features herein presented constitutes a set comprising an anatomic and orthopedic mattress (1), sufficiently stable for the backbone to be kept in an orthopedically acceptable position, provided with pillow top (2), which is an extra layer attached to the higher and/or lower part of the mattress (1), so to assure better comfort and sweetness, both covered by personalized Jacquard cloth (3) with antiallergenic, antimite and antimould treatment. The mattress (1) comprises various layers, i.e.: medium density polyurethane foams (4), agglomerated foam (5), high density polyurethane foam (6), light and undeformable expanded polypropylene or expanded polyethylene plate (7) and a natural latex foam (8), including % 0.7 of a blue dye, 52.9% of a hydrated alumina, 44.1% of calcite, 1.8% of ricinoleate and 0.5% of acrylic polymer; smooth and resistant covering, composing in this fashion a lighter structure with a lower quantity of layers and consequently less assembling stages in the production line. Pillow top (2) to be used on the higher and/or lower part of the mattress (1) comprises an extra foam layer (9) overlapped by a permeable therapeutical profiled material (10) provided with protuberances providing a smooth touch, causing body to relax, being said protuberances interspersed with magnet tablets (11) which, together with the acrylic or polyester mantle layer (12) overlaying it and which is treated and impregnated with powdered tourmaline, generate an electromagnetic force field with the capacity to naturally promote the perfect balance of body and mind health, by acting directly on blood circulation, eliminating toxins by breaking the water molecule group, causing body liquids to become weakly alkaline, sending oxygen to cells, speeding up the cell renewal process and helping to balance organ operations, combating physical fatigue, muscle pains and stress, providing deeper sleep, eliminating the daily fatigue and renewing energies. Said impregnation of powdered tourmaline on the acrylic, polyester or general cloth mantle (12) is done by mixing it to the butadiene-styrene copolymer latex added to a ricinoleate solution containing 39-45% of soap (Sulfopon®), being said mixing sprayed so to be homogeneously spread over its surface and, after drying, said mixing is impregnated, thus becoming a part of the mantle or cloth (12), which now has electromagnetic features by means of a simple process with low cost for industrial feasibility.

It should be understood that the preferred embodiments mentioned here are merely illustrative of the present invention. Numerous variations in design and use of the present invention may be contemplated in view of the following claims without straying from the intended scope and field of the invention herein disclosed.

I claim:

1. A therapeutic device with electromagnetic features comprising in combination:
an anatomic, orthopedic mattress that is sufficiently stable for the backbone to be kept in an orthopedically acceptable position;
at least one electromagnetic pillow top attached to one side of the mattress, the mattress and the pillow top both being covered by a cloth provided with antiallergenic, antimite and antimould properties;
the mattress including a medium density polyurethane foam layer, an agglomerated foam layer, a high density polyurethane foam layer, a light and undeformable plate, a natural latex foam layer, and a smooth and resistant covering; and
the pillow top including an extra foam layer overlapped by a permeable therapeutical profiled material with protuberances being interspersed with magnet tablets, the extra foam layer being overlapped by a mantle layer impregnated with powdered tourmaline.

2. The therapeutic device according to claim 1, having an additional pillow top attached to an opposite side of the mattress.

3. The therapeutic device according to claim 1, wherein the pillow top is covered by a Jacquard cloth.

4. The therapeutic device according to claim 1, wherein the mantle is composed of a material chosen from the group consisting of acrylic, polyester and cloth.

5. The therapeutic device according to claim 1, wherein the light and undeformable plate is composed of a material chosen from the group consisting of expanded polypropylene and expanded polyethylene.

6. The therapeutic device according to claim 1, wherein the natural latex foam includes:
a blue dye,
a flame retardant,
a calcite,
a ricinoleate solution, and
an acrylic polymer.

7. The therapeutic device according to claim 6, wherein the flame retardant is hydrated alumina.

8. The therapeutic device according to claim 7, wherein the natural latex foam is formed by:
0.7% of the blue dye,
52.9% of the hydrated alumina,
44.1% of the calcite,
1.8% of the ricinoleat solution, and
0.5% of the acrylic polymer.

9. The therapeutic device according to claims 1 or 8, wherein the ricinoleate solution contains 39% to 45% of soap.

10. A method for making a mantle material for a therapeutic device including an orthopedic mattress, at least one electromagnetic pillow top attached to one side of the mattress, the pillow top including an extra foam layer overlapped by a layer of mantle material impregnated with powdered tourmaline, the method comprising the steps of:
(a) mixing powdered tourmaline with butadiene-styrene copolymer latex added to a ricinoleate solution;
(b) spraying the mixture of step (a) such that the mixture is spread homogeneously over the surface of the layer of mantle; and
(c) drying the mixture being impregnated, thus becoming a part of the mantle.

11. The therapeutic device according to claim 10, wherein the ricinoleate solution contains 39% to 45% of soap.
